(19) United States
(12) Patent Application Publication
(10) Pub. No.: US 2005/0020987 A1
(43) Pub. Date: Jan. 27, 2005

(54) Syringe

(76) Inventor: Ping-Te Huang, Taichung City (TW)

Correspondence Address: ROSENBERG, KLEIN & LEE, 3458 ELLICOTT CENTER DRIVE-SUITE 101, ELLICOTT CITY, MD, 21043, US

(21) Appl. No.: 10/894,004

(22) Filed: Jul. 20, 2004

(30) Foreign Application Priority Data
Jul. 21, 2003 (TW) 92119839

(51) Int. Cl. A61M 5/31

(52) U.S. Cl. 604/240

(57) ABSTRACT

A syringe has a connector installed in a barrel. The connector has a socket and a sealing member molded on the socket directly. The socket has an exterior side and an interior and is made of a flexible material. A diameter of the sealing member is greater than a diameter of exterior side of the socket. The connector is installed in the barrel in an airtight condition. Therefore, the connector has a simple structure and reduces the numbers of the components for assembly. The syringe of the present invention is easier for assembly and the process of assembly is simplified.

### BACKGROUND OF THE INVENTION

1. Field of the Invention

The present invention relates generally to a syringe, and more particularly to syringe, which the needle is drawn back into the barrel after injection.

2. Description of the Related Art

FIG. 1 and FIG. 2 show a conventional syringe having a barrel 1, a connector 2 mounted in the barrel 1, a needle unit 3 screwed to be coupled with the connector 2 and a plunger 4 received in the barrel 1 to be pushed or drawn. The barrel 1 has a chamber 101, a hollow protrusion 102 and a wall portion 103. The hollow protrusion 102 is provided at an end of the barrel 1 with a smaller diameter and has a hole communicated with the chamber 101. The wall portion 103 is an annular wall at between the chamber 101 and the hollow protrusion 102. The connector 2 has an annular flange 201 to be against the wall portion 103, a sleeve 202 projected from the flange 201 and a O ring 203 mounted on sleeve 202. The sleeve 202 has an annular lip 204 at an interior side thereof adjacent to the flange 201 and a thread portion 205 at the interior side opposite to the lip 104. The needle unit 3 has a hub 301 having a thread portion at an exterior side to be screwed for engagement with the thread portion 205 of the connector 2 and a needle 302 with an end mounted in the hub 301. The plunger 4 has a shank 401, a rubber bulb 402 at an end of the shank 401 to be squeezed in the chamber 101 of the barrel 1 for an airtight chamber 101 and a snap 403 on the bulb 402 to be connected to connector 2. The snap 403 has a slot 404 to provide the snap 403 can be compressed and automatic rejected after the pressure has been released.

To assemble the syringe, the connector 2 is inserted into the chamber 101 of the barrel 1 via an opening at a rear end thereof, the sleeve 202 is inserted into the hollow portion 102 and the flange 201 is attached on the wall portion 103. The plunger 4 is then inserted into the chamber 101 and stopped by the connector 2 to position the connector 2. It has to be aware that the snap 403 of the plunger 4 can not run over the lip 203 of the connector 2 in this condition. And then, the needle unit 3 is screwed for connection the connector 2 via the hole of the hollow portion 102 and the syringe is completed.

The conventional syringe serves its desired function, but it still has drawbacks for fabrication and assembly. The drawbacks are described hereunder:

1. The O ring 203 is a single element to be fitted to the connector 2. It is cost much in fabrication and assembly.

2. The size of the O ring 203 is small which it is hard for labors to assemble them. While the O rings 203 are assembled by a machine, the machine might be shut down because that the elasticity of the O rings 203 make them easy to bounce out of the connector 2. If an O ring bounce into the machine, it must stop the machine for fix.

3. In assembly, it must push the plunger 2 drive the connector 2 to the desired position, and then the needle unit 3 can be screwed for connection the connector 2. It is not an easy process for assembly and, as a result, the speed of production can not increase.

4. To fit the O ring 203 to the connector 2, the components of syringe are increased and to drive the snap 403 over the lip 204 of the connector 2, the snap 403 has to be provided with the slot 404 so that the snap 403 has a complex structure.

5. The connector 2 and the needle unit 3 are two independent elements to be assembled. In assembly, the needle unit 3 has to be screwed for engagement the thread portion 205 of the connector 2. The process increases a further step of assembly.

### SUMMARY OF THE INVENTION

The primary objective of the present invention is to provide a syringe, which is easier and faster to assemble and while it is assembled by a machine, it prevents elements from bouncing to increase the capacity of fabrication.

The secondary objective of the present invention is to provide a syringe, which the needle is drawn back into the barrel after injection and the connector drives the needle tilting to prevent the needle from extruding out of the barrel.

According to the objectives of the present invention, a syringe comprises a barrel having a barrel member and a beam tube. The barrel member has an axis and a chamber. The beam tube is smaller then the barrel member and has a through hole, which a diameter thereof is smaller than a diameter of the chamber, communicated with the chamber. A stop portion is formed at between the chamber and the through hole. A connector is installed in the chamber of the barrel via an opening opposite to the beam tube and attached on the stop portion. The connector has a socket and a sealing member molded on the socket directly. The socket has an exterior side surrounding the axis and an interior side opposite to the exterior side on which a first lock portion is projected and a receiving portion formed beside the first lock portion and distal to the axis than the first lock portion. The sealing member is made of a flexible material and is mounted on the exterior side of the socket. A diameter of the sealing member is greater than a diameter of the exterior side of the socket. A needle unit has a hub and a needle mounted on the hub. The hub is connected to the socket of the connector, and a plunger is inserted into the chamber of the barrel for reciprocation. The plunger has a shank for operation, a bulb mounted at an end of the shank to provide chamber an airtight condition and a snap mounted on the snap adapted to cross the first lock portion and rest in the receiving portion.

### BRIEF DESCRIPTION OF THE DRAWINGS

FIG. 1 is an exploded view of the conventional syringe;

FIG. 2 is a sectional view of the conventional syringe in combination;

FIG. 3 is an exploded view of a first preferred embodiment of the present invention;

FIG. 4 is a sectional view along the 4-4 line of FIG. 3;

FIG. 5 is a sectional view of the first preferred embodiment of the present invention, showing the connector connected to the needle unit and both of them installed in the barrel;

FIG. 6 is a sectional view of the first preferred embodiment of the present invention, showing the plunger driving the snap engaged with the connector;

FIG. 7 is a sectional view of the first preferred embodiment of the present invention, showing the plunger being drawn to draw the needle into the barrel;

FIG. 8 is a sectional view in part of a second preferred embodiment of the present invention, showing the connector;

FIG. 9 is a sectional view of the second preferred embodiment of the present invention, showing the plunger being drawn to draw the needle into the barrel;

FIG. 10 is a sectional view in part of a third preferred embodiment of the present invention;

FIG. 11 is a sectional view in part of a fourth preferred embodiment of the present invention.

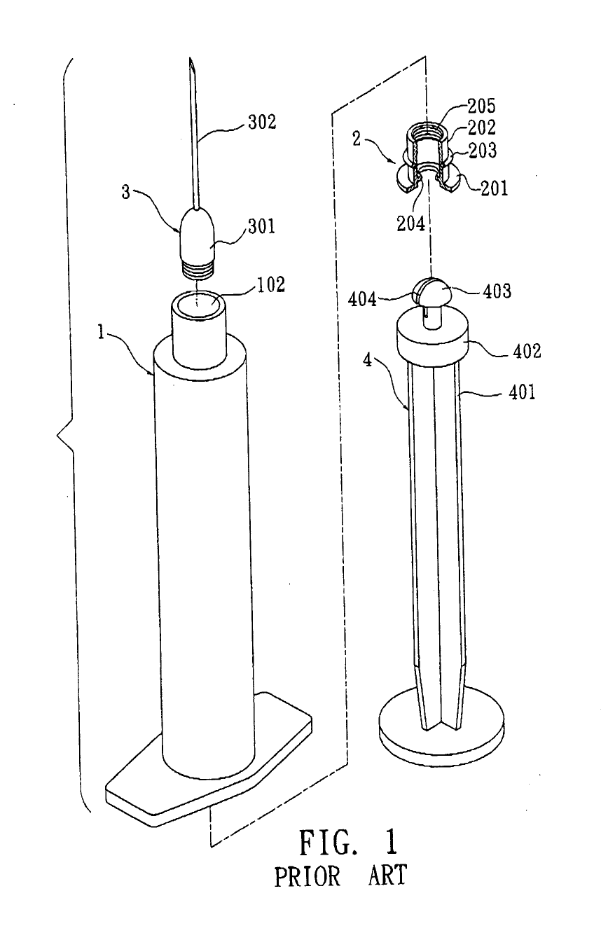

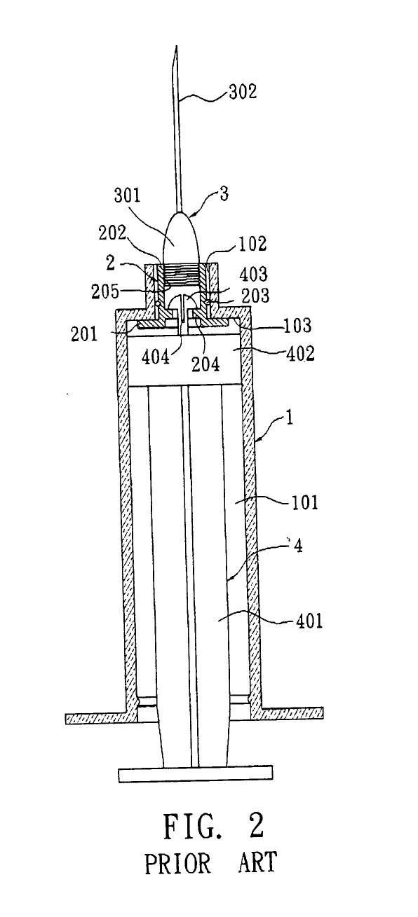

### DETAILED DESCRIPTION OF THE INVENTION

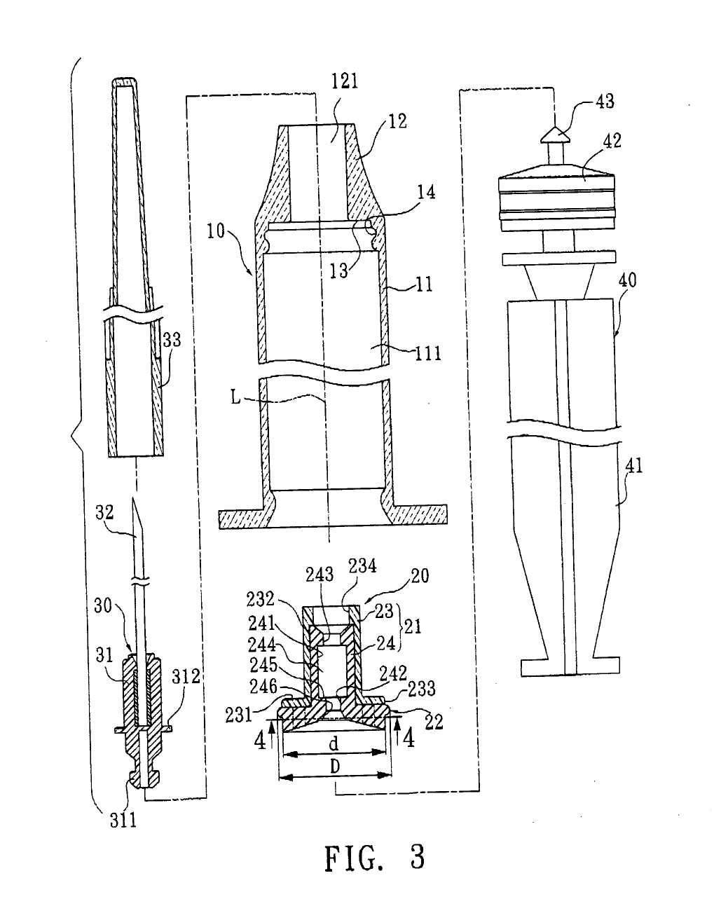

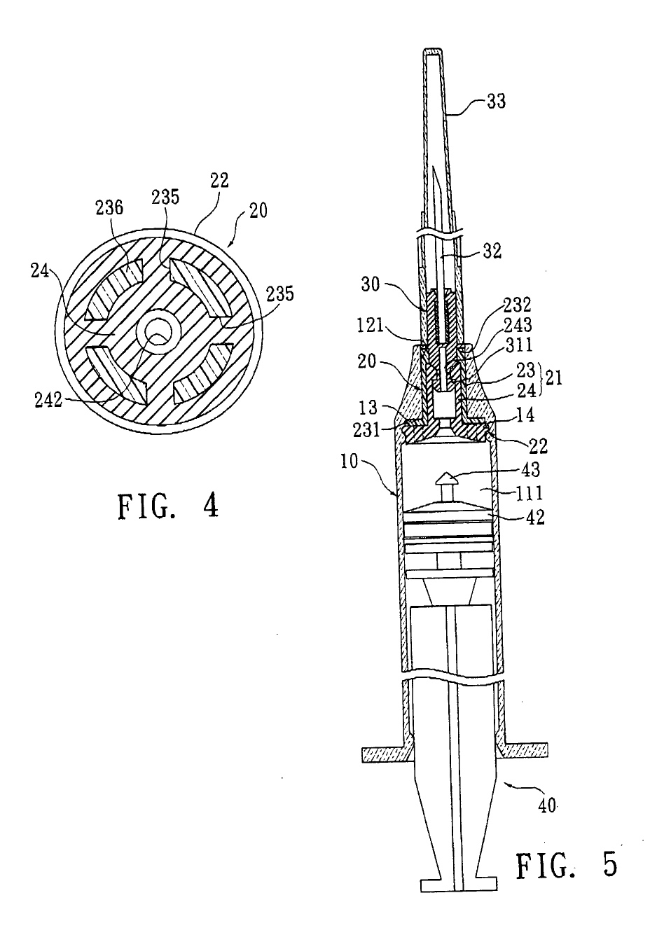

As shown in FIGS. from FIG. 3, FIG. 4 and FIG. 5, a syringe of the first preferred embodiment of the present invention comprises a barrel 10, a connector 20 mounted in the barrel 10, a needle unit 30 connected to the connector 20 and a plunger 30 inserted into the barrel 10 to be pushed and drawn.

The barrel 10 is a hollow member having a barrel member 1, which has an axis L and a chamber 111 inside, and a beam tube 12 in which a through hole 121 communicated with the chamber 111. A diameter of the through hole 121 is smaller than a diameter of the chamber 111. The barrel 10 has a stop portion 13 at between the chamber 111 and the through hole 121 and an annular slot 14 beside the stop portion 13.

The connector 20 is a two-order hollow cylinder and is inserted into the chamber 111 of the barrel 10 via an opening of the barrel member 10 at an end opposite to the beam tube 12. The connector 20 has a socket 21 and a sealing device 22. It is noted that the socket 21 and the sealing member 22 are molded into a single element. The socket 21 has an outer socket 23, which is made of a rigid plastic, such as polypropylene (PP), and an inner socket 24, which is made of a flexible or elastic material, such as rubber. The inner socket 24 is mounted at an interior side of the outer socket 23. The outer socket has an annular flange 231, a sleeve 232, an exterior side 233 and an interior side 234. The flange 231 is adapted to be against the stop portion 13. The sleeve 232 is projected from the flange 231 and has a diameter smaller than the flange 231. The exterior side 233 surrounds the axis L and the interior side 234 is opposite to the exterior side 233. The flange 231 is provided with a plurality of radial tunnels 235 and blocks 236 between the neighboring tunnels 235 respectively. The interior socket 24 and the sealing member 22 are molded into a single member, wherein the sealing member 22 surrounds the interior socket 24 at an exterior side thereof. The interior socket 24 has an annular interior side 241 surrounding the axis L on which a first lock portion 242 and a second lock portion 243 are projected inwardly, The first lock portion 242 is proximal to the chamber 111 and the second lock portion 243 is opposite to the first lock portion 242. The interior side 241 of the interior socket 24 further has a receiving portion 244 between the first lock portion 242 and the second lock portion 243, a stop portion 245 between the first lock portion 242 and the receiving portion 244 and a guiding portion 245 proximal to the chamber 111 and at a side of the first lock portion 242. The guiding portion 246 is a conical hole. The sealing member 22 is made of a flexible or elastic material, such as rubber and is molded on the flange 231 of the outer socket 23 via the tunnels 235 directly to be firmly coupled with the flange 231 and the inner socket 24. The blocks 236 restrict the inner socket 24 at a predetermined position. An exterior portion of the sealing member 22 is extruded out of the exterior side 233 and an interior portion thereof is molded on the inner socket 24 directly. The sealing member 22 has a diameter D greater than a diameter d of the outer side 233 of the outer socket 23.

The needle unit 30 has a hub 31, a needle 32 mounted on the hub 31 and a lid 33 fitted to the hub 31 to shield the needle 32. The hub 31 has a connector portion 311 on an exterior side thereof and a lock ring 312. The connector portion 311 is able to be inserted into the through hole 121 and cross the second lock portion 243 and rest in the receiving portion 244. The lock ring 312 is against the sleeve 232 to be lock with the lid 33.

The plunger 40 is inserted into the chamber 111 of the barrel 10 having a shank 41 for operation, a rubber bulb 42 at an end of the shank 41 and a snap 43 projected from the bulb 42. The bulb 42 provides the chamber 111 an airtight condition. The snap 42 is able to cross the first lock portion 242 and be locked in the receiving portion 244. With the material property of the interior socket 24, after the snap 24 has crossed the first lock portion 242, it is firmly connected to the connector 20.

For assembly, as shown in FIG. 5, the connector portion 311 of the needle unit 30 is inserted into the sleeve 232 of the connector 20. Because of the material property of the inner socket 24, the connector portion 311 can cross the second lock portion 243 easily to firmly couple the needle unit 30 with the connector 20. And then, the connector 20 and the needle unit 30 with the lid 33 is inserted into the chamber 111 via the opening thereof to align the needle 32 and the lid 33 to the through hole 121. While the sealing member 22 has been engaged with the annular slot 14 and the flange 231 has been attached on the stop portion 13, the connector 20 and the needle unit 30 are fixed in the barrel 10. At last, the bulb 42 and the snap 43 of the plunger 40 are inserted into the chamber 111 to complete the syringe of the present invention. The process of assembly as described above can be taken by a machine and the sealing member will not depart from the socket 21.

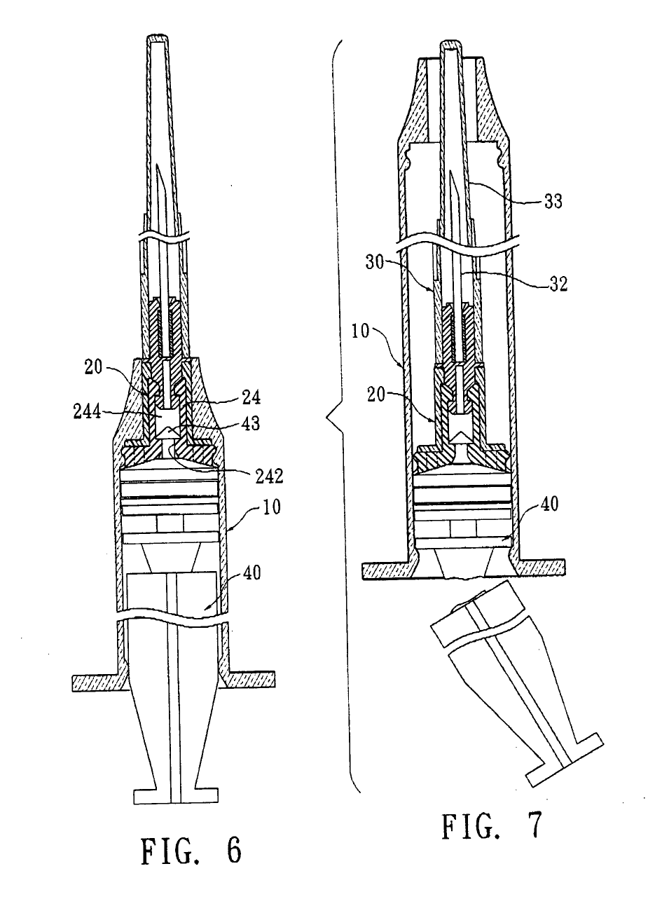

As shown in FIG. 6, after the injection had taken and the syringe is going to throw away, user just has to push the plunger 40 inwardly. Because of the material property of the inner socket 24, the snap 43 can cross the first lock portion 242 easily and go to the receiving portion 244. As a result, the plunger 40 is coupled with the connector 20. And then, as shown in FIG. 7, user draws the plunger 40 until the connector 20 and the needle unit 30 totally received in the barrel 10. After that user can break the plunger 40 to prevent the needle 32 from hurting people.

The advantages of the syringe of the first preferred embodiment of the present invention are:

1. The sealing member 22 is molded on the socket 21 directly, which is fabricated totally by a machine without any labor involved. It reduces the cost of fabrication and assembly.

2. The sealing member 22 is molded on the socket 21 directly, which is fabricated totally by a machine. It does not need labor to assemble, such that the cost of assembly is reduce. The process of assembling the connector 20 to the syringe can be taken by a machine and the sealing member 22 will not bounce. The process of assembly has a well reliability and it has less ratio of defect, less chance to shot down the machine and fast assembly speed to increase the capacity if production.

3. In assembly, the connector 20 is connected to the needle unit 30 prior to the connector 20 and the needle unit 30 are installed in the barrel 10. It has a easier way of assembly and the speed of assembly is increased to increase the capacity if production.

4. The sealing member 22 is molded on the socket 21 directly to reduce the numbers of the components. Because of the material property of the inner socket 24, it has no need to provide slot on the snap 43 so that the snap 43 has a simple structure. The process of assembly can be taken in a single machine, at the same time and in a single step.

5. As shown in FIG. 4, the outer socket 23, which is made of polypropylene, and the blocks 236 supports the inner socket 24 and the sealing member 22, which are made of rubber. It provides the snap 43 of the plunger 40 connected to the connector in a firm condition and in a tight fastening condition.

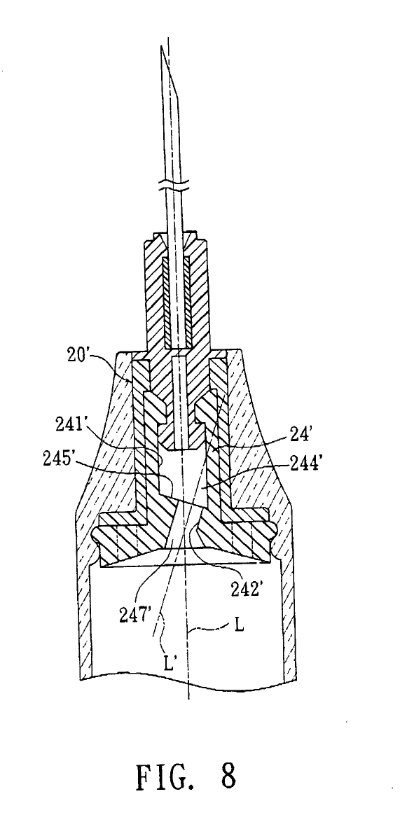

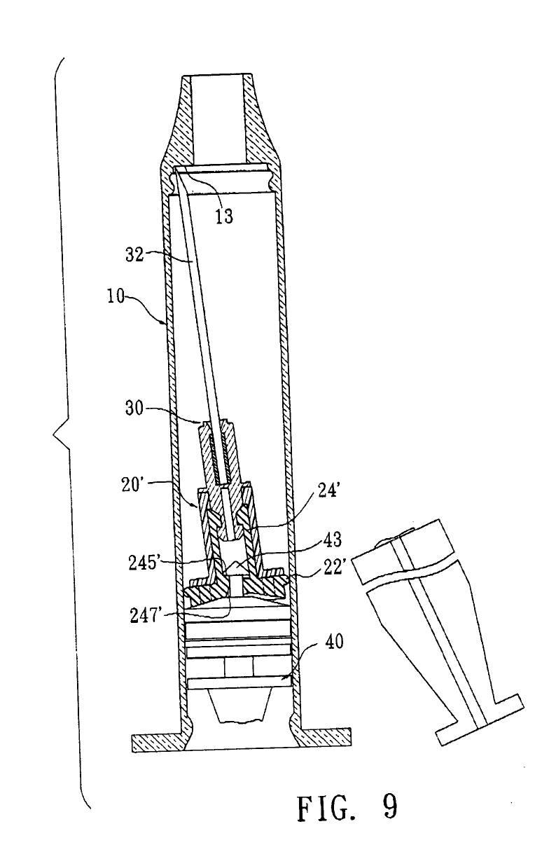

As shown in FIG. 8, a syringe of the second preferred embodiment of the present invention, which is similar to the syringe of the first preferred embodiment, except that a connector 20′ has a bias lip 245′ on an interior side 241′ of an inner socket 24′. The bias lip 245′ is located between a first lock portion 242′ and a receiving portion 244′. The first lock portion 242′ has a bias hole 247′ along a bias axis L′ which the bias axis L′ inclines to an axis L about 20 degrees and the bias axis L′ is perpendicular to the bias lip 245′. As shown in FIG. 9, while a plunger 40 is connected to the connector 20′ via a snap 43, the inner socket 24′ is exerted by a lateral force by the snap 43. In the meantime, the sealing member 22′ is restricted by the barrel 10 because it presses the barrel 10. While a plunger 40 is drawn (before that a lid can be taken off) to draw the connector 2 and the needle unit 30 into the barrel 10, the lateral force exerted on the inner socket 24′ is gone to tilt the connector 20′ and the needle unit 30. Finally, the plunger 40 is broken. In this condition, the needle 32 of the needle unit 30 is stop by a stop portion 13 of the barrel 10 to prevent it from escape from the barrel 10 again.

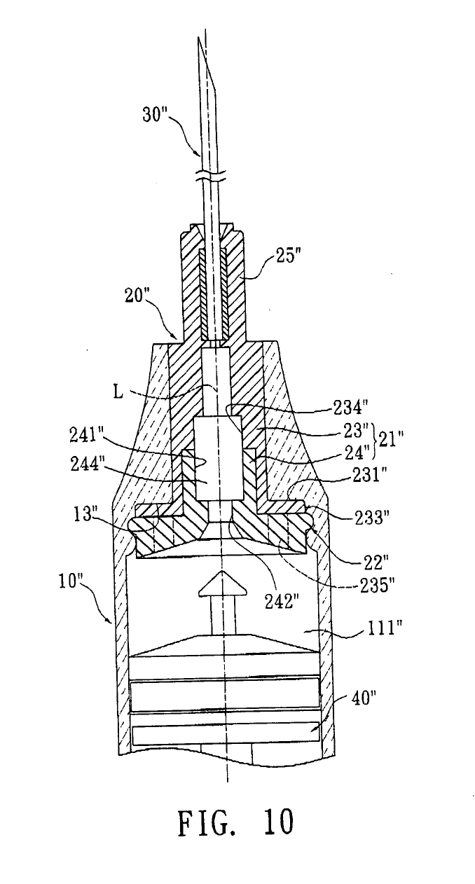

FIG. 10 shows a syringe of the third preferred embodiment of the present invention, which comprises a barrel 10″, a connector 20″ installed in the barrel 10″, a needle 30″ mounted on the connector 20″ and a plunger 40″ inserted into the barrel 10″ for reciprocation. The barrel 10″ and the plunger 40″ are as same as the barrel 10 and the plunger 40 of the first preferred embodiment. Hereunder we only describe the detail of the connector 20″ and the needle 30′.

The connector 20″ has a socket 21″ on which a sealing member 22″ and a hub 25″ are molded directly. The socket 21″ has an outer socket 23″, which is made of a rigid plastic material, and an inner socket 24″, which is made of a flexible or elastic material, mounted in the outer socket 23″. The outer socket 23″ has an annular flange 231″, a sleeve 232″, an exterior side 233″ and an interior side 234″. The flange 231″ is adapted to be attached on a stop portion 13″ of the barrel 10″. The sleeve 232″ is projected from the flange 231″ having a diameter smaller than flange 231″. The exterior side 233″ is located at an exterior of the flange 231″ and surrounds an axis L and the interior side 234″ is opposite to the exterior side 233″. The flange 231″ has a plurality of tunnels 235″ along its radial directions. The inner socket 24″ has an interior side 241″ surrounding the axis L on which a lock portion 242″ and a receiving portion 243″ are provided. The lock portion 242″ is projected from the interior side 241″ and is proximal to a chamber 111″ of the barrel 10″. The receiving portion 243″ is located at beside the lock portion 242″ and is distal to the axis L than the lock portion 242″. The sealing member 22″ is made of a rubber material to be molded in the tunnels 244″, which has an exterior portion extruded out of the exterior side 233″ and an interior portion molded on the inner socket 24″ directly. The sealing member 22″ has a diameter greater than a diameter of the exterior side 233″ of the outer socket 23″.

The needle 30″ is mounted to the hub 25″ of the connector 20″ and is extruded out of the barrel 10″

The syringe of the third preferred embodiment provides the socket 21″, the sealing member 22″ and the hub 25″ molded into a single component to reduce the numbers of the components of the syringe and to simplify the fabrication and assembly. It only needs to insert the needle 30″ into the hub 25″. To compare with the conventional syringe which the needle unit is screwed to fasten it to the connector, the syringe of the third preferred embodiment provides the results of reducing the numbers of components and simplifying the process of assembly.

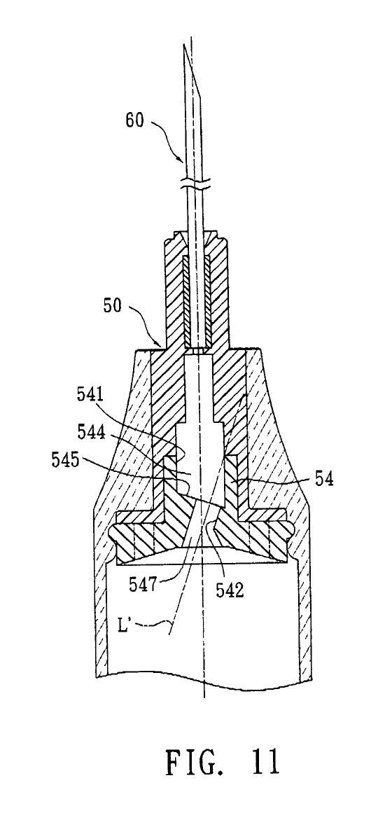

FIG. 11 shows a syringe of the third preferred embodiment of the present invention, which is similar to the syringe of the third preferred embodiment, except that a connector 50 has an inner socket 54 and a bias lip 545 is provided on an interior side 541 of the inner socket 54 between a lock portion 542 and a receiving portion 544. The inner socket 54 further has a bias hole 547 in the lock portion 542 surrounding a bias axis L′. The bias axis L′ is perpendicular to the bias lip 545. While the connector 50 and a needle 60 are drawn into a barrel, it prevents the needle 60 from escape from the barrel, which the needle is similar to the condition shown in FIG. 9.

In conclusion, the present invention provides the syringe with the Sealing member molded on the socket directly. This achieves the functions of sealing the barrel, simplifying the fabrication and assembly, increasing the capacity of production and reducing the cost.

In the specification of the present invention, we only provide a few of preferred embodiments. The scope of the present invention should not be restricted in the preferred embodiment. Any equivalent designation should be still in the scope of the present invention.

1. A syringe, comprising:
a barrel having a barrel member and a beam tube, wherein the barrel member has an axis and a chamber, the beam tube is smaller then the barrel member and has a through hole, which a diameter thereof is smaller than a diameter of the chamber, communicated with the chamber and a stop portion is formed at between the chamber and the through hole;
a connector installed in the chamber of the barrel via an opening opposite to the beam tube and attached on the stop portion, which has a socket and a sealing member molded on the socket directly, wherein the socket has an exterior side surrounding the axis and an interior side opposite to the exterior side on which a first lock portion is projected and a receiving portion is formed beside the first lock portion, which is distal to the axis than the first lock portion, and the sealing member is made of a flexible material and is mounted on the exterior side of the socket, wherein a diameter of the sealing member is greater than a diameter of the exterior side of the socket;
a needle unit having a hub and a needle mounted on the hub, wherein the hub is connected to the socket of the connector, and
a plunger inserted into the chamber of the barrel for reciprocation and having a shank for operation, a bulb mounted at an end of the shank to provide chamber an airtight condition and a snap mounted on the snap adapted to cross the first lock portion and rest in the receiving portion.

2. The syringe as defined in claim 1, wherein the socket of the connector is made of polypropylene and the sealing member is made of rubber.

3. The syringe as defined in claim 1, wherein the socket of the connector has an exterior socket and a flexible interior socket, on an exterior side of which the sealing member is formed and on an interior side of which the first lock portion and the receiving portion are formed.

4. The syringe as defined in claim 3, wherein the connector further has a bias lip on the interior side of the inner socket between the first lock portion and the receiving portion and a bias hole in the first lock portion along a bias axis, wherein the bias axis inclines to the axis for a predetermined angle and the bias axis is perpendicular to the bias lip.

5. The syringe as defined in claim 3, wherein the outer socket of the socket of the connector is made of polypropylene and the inner socket is made of rubber.

6. The syringe as defined in claim 3, wherein the inner socket of the connector further has a guiding portion, which is a cone hole located beside the first lock portion and proximal to the chamber.

7. The syringe as defined in claim 3, wherein the outer socket of the connector has a plurality of tunnels, in which a flexible material flows therethrough to mold the interior socket and the sealing member, and blocks at between the tunnels respectively.

8. The syringe as defined in claim 1, wherein the socket of the connector has an outer socket and a flexible inner socket, on an exterior side of which the sealing member is molded and a second lock portion is projected, and the receiving portion is located at between the first lock portion and the second lock portion and the hub of the needle member has a connector portion adapted to cross the second lock portion and rest in the receiving portion.

9. A syringe, comprising:
a barrel having a barrel member and a beam tube, wherein the barrel member has an axis and a chamber, the beam tube is smaller then the barrel member and has a through hole, which a diameter thereof is smaller than a diameter of the chamber, communicated with the chamber and a stop portion is formed at between the chamber and the through hole;
a connector installed in the chamber of the barrel via an opening opposite to the beam tube and attached on the stop portion, which has a socket, a sealing member and a hub molded on the socket directly, wherein the socket has an exterior side surrounding the axis and an interior side opposite to the exterior side, on which a lock portion is projected and a receiving portion is formed beside the lock portion and distal to the axis than the lock portion, and the sealing member is made of a flexible material and is mounted on the exterior side of the socket, wherein a diameter of the sealing member is greater than a diameter of the exterior side of the socket;
a needle mounted in the hub and extruded out of the barrel, and
a plunger inserted into the chamber of the barrel for reciprocation and having a shank for operation, a bulb mounted at an end of the shank to provide chamber an airtight condition and a snap mounted on the snap adapted to cross the lock portion and rest in the receiving portion.

10. The syringe as defined in claim 9, wherein the socket of the connector is made of polypropylene and the sealing member is made of rubber.

11. The syringe as defined in claim 9, wherein the socket of the connector has an exterior socket and a flexible interior socket, on an exterior side of which the sealing member is formed and on an interior side of which the lock portion and the receiving portion are formed.

12. The syringe as defined in claim 11, wherein the outer socket of the socket of the connector is made of polypropylene and the inner socket is made of rubber.

13. The syringe as defined in claim 11, wherein the outer socket of the connector has a plurality of tunnels, in which a flexible material flows therethrough to mold the interior socket, and blocks at between the tunnels respectively.

14. The syringe as defined in claim 9, wherein the inner socket of the connector further has a guiding portion, which is a cone hole located beside the lock portion and proximal to the chamber.

15. The syringe as defined in claim 9, wherein the connector further has a bias lip on the interior side of the inner socket between the lock portion and the receiving portion and a bias hole in the lock portion along a bias axis, wherein the bias axis inclines to the axis for a predetermined angle and the bias axis is perpendicular to the bias lip.
